(19) United States
(12) Patent Application Publication
(10) Pub. No.: US 2024/0352401 A1
(43) Pub. Date: Oct. 24, 2024

(54) FURFURAL-RESISTANT STRAIN AND METHOD FOR IMPROVING FURFURAL-RESISTANT OF STRAIN

(71) Applicant: Korea Institute of Science and Technology, Seoul (KR)

(72) Inventors: Sun Mi LEE, Seoul (KR); Jiwon Kim, Seoul (KR); Hyeoncheol Son, Seoul (KR); Ja Kyong Ko, Seoul (KR); Gyeongtaek Gong, Seoul (KR); Jung Ho Ahn, Seoul (KR); Youngsoon Um, Seoul (KR)

(21) Appl. No.: 18/521,797

(22) Filed: Nov. 28, 2023

(30) Foreign Application Priority Data
Apr. 21, 2023 (KR) 10-2023-0052719

(51) Int. Cl. C12N 1/16; C12N 9/02; C12N 15/81

(52) U.S. Cl. CPC C12N 1/165; C12N 9/0008; C12N 15/815; C12R 2001/645; C12Y 102/01003

(57) ABSTRACT

The present disclosure discloses a strain having resistance by neutralizing furfural, an inhibitor derived from lignocellulosic biomass, based on a technique using aldehyde dehydrogenase and a method of using the same. According to the present disclosure, furfural, an inhibitor derived from lignocellulosic biomass, is neutralized based on a technique using aldehyde dehydrogenase, so that a resistant strain overexpresses aldehyde dehydrogenase (EcAldH) in Yarrowia lipolytica, which has the ability to produce yeast oil, and has resistance to furfural, a biomass-derived inhibitor. Accordingly, even in the medium containing furfural, it can provide excellent strain growth and yeast oil production ability.

### CROSS REFERENCE TO RELATED APPLICATION

The present application claims priority to Korean Patent Application No. 10-2023-0052719, filed Apr. 21, 2023, the entire contents of which is incorporated herein for all purposes by this reference.

### BACKGROUND OF THE INVENTION

### Field of the Invention

A novel strain having furfural neutralization ability and resistance is disclosed herein.

### DESCRIPTION OF GOVERNMENT-SPONSORED RESEARCH

This study was conducted by the following national project.
Ministry Name: Ministry of Science and ICT; Project management (specialized) institute name: Korea Institute of Science and Technology; Research program title: Korea Institute of Science and Technology research operation cost support (main project cost); Research project title: Original technology development for electro super cellulose composite material; Project number: 2E32520; Project assignment number: 1711196516
Ministry Name: Ministry of Strategy and Finance; Project management (specialized) institute name: National Research Foundation; Research program name: Climate change mitigation technology development; Research project name: Development of strain and bioprocess for conversion of unused biomass to produce medium/long chain fatty acids; Project number: 2020M1A2A2080847; Project assignment number: 1711158983

### Description of the Related Art

As concerns about the acceleration of global warming caused by the continuous use of fossil fuels, environmental pollution problems, and the possibility of petroleum resource depletion are rising, the priority of developing sustainable alternative energy sources is greatly rising throughout the industry. Among alternative energy sources, biofuel produced through the cultivation of microbial strains is attracting attention as an alternative. When using biofuel, it is emphasized that existing infrastructure can be used as it is without the need to build new industrial facilities, making it a realistic and efficient alternative. As a raw material of the biofuel, lignocellulosic biomass is widely used, and it is composed of cellulose, hemicellulose, lignin, etc. Among them, cellulose and hemicellulose can be converted into glucose or xylose sugar, which are necessary resources for microorganisms, through a pretreatment process.

The pretreatment process of lignocellulosic biomass is a process of decomposing cellulose components through high temperature and high pressure to make sugar. In the process of strong acid treatment at high temperature and pressure, not only glucose and xylose sugar but also furfural and hydroxymethylfurfural are simultaneously generated. Since these by-products generally act as toxic substances to microorganisms, if the hydrolysate made from lignocellulosic biomass contains the corresponding by-products, the growth of microorganisms is inhibited, which leads to a decrease in the productivity of biofuels.

Among various by-products, furfural derived from xylose sugar is pointed out as one of the most toxic inhibitors. In particular, it was confirmed that the Yarrowia lipolytica strain, which shows high lipid productivity in biofuel production, is significantly inhibited in cell growth even at a very low concentration (˜0.5 g/L) of furfural.

As a method for neutralizing furfural in general yeast strains such as Saccharomyces cerevisiae, a method using alcohol dehydrogenase is generally used. However, in some oleaginous yeasts, comprising Yarrowia lipolytica, the method using alcohol dehydrogenase cannot obtain a significant effect in many cases due to differences in the internal neutralization mechanism. Therefore, it is necessary to develop new technologies in order to enhance the furfural resistance of Yarrowia lipolytica.

### SUMMARY OF THE INVENTION

The Sequence Listing created on Apr. 21, 2023 with a file size of 48.00 KB, and filed herewith in XML file format as the file entitled “Sequence_Listing-457KCL0056US,” is hereby incorporated by reference in its entirety.

In one aspect, the problem to be solved by the present disclosure is to provide a Yarrowia lipolytica transformant strain with enhanced furfural resistance.

In another aspect, the problem to be solved by the present disclosure is to provide a yeast oil production method having excellent productivity through fermentation of a Yarrowia lipolytica transformant strain having enhanced furfural resistance.

In another aspect, the problem to be solved by the present disclosure is to provide a furfural neutralization method using a Yarrowia lipolytica transformant strain having enhanced furfural resistance.

In order to achieve the above object, one embodiment of the present disclosure provides a furfural-resistant strain comprising a gene encoding an aldehyde dehydrogenase (EcAldH) gene, wherein the aldehyde dehydrogenase gene comprises at least one gene sequence selected from the group consisting of EcAldH (PuuC) having a nucleotide sequence of SEQ ID NO: 1; YALI0A17875g having a nucleotide sequence of SEQ ID NO: 2; YALI0E15400g having a nucleotide sequence of SEQ ID NO: 3; YALI0B01298g having a nucleotide sequence of SEQ ID NO: 4; and YALI0F23793g having a nucleotide sequence of SEQ ID NO: 5.

One embodiment of the present disclosure provides a method for producing yeast oil comprising culturing the above-described furfural-resistant strain in a medium containing furfural.

One embodiment of the present disclosure provides a method for neutralizing furfural, comprising converting furfural into 2-furoic acid by culturing the furfural-resistant strain in a medium containing furfural.

In one aspect, the present disclosure relates to a strain having resistance by neutralizing furfural, an inhibitor derived from lignocellulosic biomass, based on a technique using aldehyde dehydrogenase and a method of using the same. According to the present disclosure, it is possible to provide a resistant strain by neutralizing furfural, an inhibitor derived from lignocellulosic biomass, based on a technique using aldehyde dehydrogenase. When aldehyde dehydrogenase (EcAldH) is overexpressed in Yarrowia lipolytica, which has the ability to produce yeast oil, to provide resistance to furfural, a biomass-derived inhibitor, the ability of strain growth and produce yeast oil is excellent even in a medium containing furfural. Therefore, it can solve the problem in which furfural resistance is enhanced when alcohol dehydrogenase is overexpressed in common yeast strains such as Saccharomyces cerevisiae, but this resistance enhancement is not observed in Yarrowia lipolytica.

### BRIEF DESCRIPTION OF THE DRAWINGS

FIG. 1A is a diagram showing cell growth of wild type Yarrowia lipolytica strain in media supplemented with various concentrations of furfural (0, 0.1, 0.2, 0.3, 0.4 and 0.5 g/L).

FIG. 1B is a diagram showing glucose concentration over time of wild type Yarrowia lipolytica strain in media supplemented with various concentrations of furfural (0, 0.1, 0.2, 0.3, 0.4 and 0.5 g/L).

FIG. 2A is a diagram showing a graph comparing relative growth rates (relative cell growth) of strains transformed with alcohol dehydrogenase (ScADH7p) or aldehyde reductase (ScOSI1p) and a negative control (wild type Yarrowia lipolytica strain) in a medium supplemented with 0.4 g/L of furfural.

FIG. 2B is a diagram showing a graph comparing relative glucose consumptions of strains transformed with alcohol dehydrogenase (ScADH7p) or aldehyde reductase (ScOSI1p) and a negative control (wild type Yarrowia lipolytica strain) in a medium supplemented with 0.4 g/L of furfural.

FIG. 3 is a diagram showing that furfural is converted to furoic acid according to the cultivation of a wild type strain of Yarrowia lipolytica, as the analysis result of the concentrations of furfural and furoic acid at the time (0 h) of first inoculation of a strain in a medium containing 0.4 g/L of furfural and at the time (120 h) of incubation for 120 hours after inoculation.

FIG. 4A is a diagram showing a graph comparing cell growths of a strain transformed with aldehyde dehydrogenase (EcAldH, YALI0F0444g, YALI0E00264g, YALI0D07942g, YALI0B01298g, or YALI0E15400g) and a negative control in a medium supplemented with 0.4 g/L of furfural at a reference time point of 72 hours incubation.

FIG. 4B is a diagram showing a graph comparing glucose consumption rates of a strain transformed with aldehyde dehydrogenase (EcAldH, YALI0F0444g, YALI0E00264g, YALI0D07942g, YALI0B01298g, or YALI0E15400g) and a negative control in a medium supplemented with 0.4 g/L of furfural at a reference time point of 72 hours incubation.

FIG. 5A is a diagram showing a graph comparing cell growth rates and glucose consumption rates of a strain transformed with aldehyde dehydrogenase (EcAldH, YALI0A17675g, YALI0E15400g, YALI0B01298g, or YALI0F23793g) and a negative control (a wild type Yarrowia lipolytica strain) in a medium supplemented with 0.4 g/L of furfural.

FIG. 5B is a diagram showing a graph comparing glucose consumption rates of a strain transformed with aldehyde dehydrogenase (EcAldH, YALI0A17675g, YALI0E15400g, YALI0B01298g, or YALI0F23793g) and a negative control (a wild type Yarrowia lipolytica strain) in a medium supplemented with 0.4 g/L of furfural.

FIG. 6A is a diagram showing a graph comparing furfural reduction rates of a strain transformed with aldehyde dehydrogenase (EcAldH, YALI0A17675g, YALI0E15400g, YALI0B01298g, or YALI0F23793g) and a negative control (a wild type Yarrowia lipolytica strain) in a medium supplemented with 0.4 g/L of furfural.

FIG. 6B is a diagram showing a graph comparing furoic acid production rates of a strain transformed with aldehyde dehydrogenase (EcAldH, YALI0A17675g, YALI0E15400g, YALI0B01298g, or YALI0F23793g) and a negative control (a wild type Yarrowia lipolytica strain) in a medium supplemented with 0.4 g/L of furfural.

FIG. 7 is a diagram confirming yeast oil production performance in Yarrowia lipolytica WF and Yarrowia lipolytica LF, which are strains transformed with YALI0E15400g among aldehyde dehydrogenases according to one embodiment of the present disclosure through fluorescence intensity measurement.

### DETAILED DESCRIPTION OF THE INVENTION

Hereinafter, preferred embodiments of the present disclosure will be described in detail with reference to the accompanying drawings.

Embodiments of the present disclosure disclosed herein are illustrated for purposes of illustration only, and the embodiments of the present disclosure may be implemented in various forms and should not be construed as being limited to the embodiments described herein. The present disclosure may have various changes and various forms, and it should be understood that the embodiments are not intended to limit the present disclosure to a specific disclosure form, and include all modifications, equivalents, and substitutes included in the spirit and scope of the present disclosure. The singular forms are intended to include the plural forms as well, unless the context clearly indicates otherwise. The terms “comprises,” “contains,” “includes,” or “having,” when used herein, specify the presence of stated features, integers, steps, operations, elements, components, or combination thereof, but do not preclude the presence or addition of one or more other features, integers, steps, operations, elements, components, or combination thereof.

In the present specification, ‘furfural’ is an organic compound mainly formed through transformation of xylose sugar, and has a chemical formula of C₄H₃OCHO and a structure of Formula 1 below.

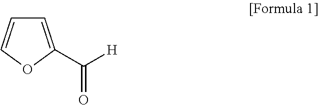

The furfural is comprised as a by-product of the process of pretreatment to make lignocellulosic biomass into a hydrolysate, and is toxic to cell growth, thereby reducing the growth of microorganisms and productivity of biofuel.

Accordingly, the present disclosure provides a strain having furfural neutralization ability and resistance through transformation. Specifically, one embodiment of the present disclosure may provide a furfural-resistant strain comprising a gene encoding an aldehyde dehydrogenase (EcAldH) gene. More specifically, the aldehyde dehydrogenase gene may comprise at least one gene sequence selected from the group consisting of EcAldH (PuuC) having a nucleotide sequence of SEQ ID NO: 1; YALI0A17875g having a nucleotide sequence of SEQ ID NO: 2; YALI0E15400g having a nucleotide sequence of SEQ ID NO: 3; YALI0B01298g having a nucleotide sequence of SEQ ID NO: 4; and YALI0F23793g having a nucleotide sequence of SEQ ID NO: 5.

In one embodiment, the furfural-resistant strain may be a transformant strain to comprise a gene encoding an aldehyde dehydrogenase gene using Yarrowia lipolytica as a parent strain. Yarrowia lipolytica is a representative lipid-producing yeast strain, which can be used for the production of various high value-added substances comprising yeast oil and β-carotene. However, in the case of wild type strain of Yarrowia lipolytica that does not express aldehyde dehydrogenase, it is difficult to grow due to various inhibitors, especially furfural, contained in the hydrolysate derived from lignocellulosic biomass. However, the strain according to an embodiment of the present disclosure acquires furfural resistance through the expression of aldehyde dehydrogenase through introduction of the said gene, and thus can grow effectively even in a hydrolysate derived from lignocellulosic biomass.

Specifically, referring to the reaction scheme of Formula 2 below, in one embodiment, the aldehyde dehydrogenase (ALDH) gene converts furfural into 2-furoic acid, which is a relatively less toxic substance, to neutralize furfural within the furfural-resistant strain, so that the strain can grow even in an environment containing furfural.

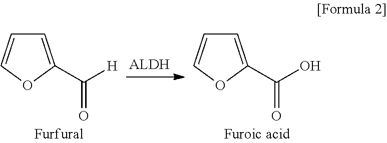 [SMILES: O=Cc1ccco1 ; O=C(O)c1ccco1]

In addition, due to the introduction of the aldehyde dehydrogenase (EcAldH) gene, the furfural-resistant strain according to an embodiment of the present disclosure can solve the problem in which furfural resistance is enhanced when alcohol dehydrogenase is overexpressed in common yeast strains such as Saccharomyces cerevisiae, but this resistance enhancement is not observed in Yarrowia lipolytica.

In one embodiment, the parent strain to be transformed may be a wild-type Yarrowia lipolytica strain. In one embodiment, the wild-type Yarrowia lipolytica strain may be a commercially available one or the one deposited in a reliable depository authority and for which the fact that it can be freely purchased through a catalog, etc. issued by the depository authority has been verified. In one embodiment, the wild-type Yarrowia lipolytica strain may be Yarrowia lipolytica PO1f strains having accession number ATCC MYA-2613, but is not limited thereto and any wild-type Yarrowia lipolytica strain may be comprised.

In another embodiment, the parent strain to be transformed may be a strain transformed with a DGA1 expression vector consisting of the nucleotide sequence of SEQ ID NO: 10 in a wild-type Yarrowia lipolytica strain.

In one embodiment, a transformant strain to comprise a gene encoding an aldehyde dehydrogenase gene using Yarrowia lipolytica as a parent strain may be obtained by expressing one selected from the group consisting of the aldehyde dehydrogenase gene (EcAldH (PuuC)) having a nucleotide sequence of SEQ ID NO: 1; YALI0A17875g having a nucleotide sequence of SEQ ID NO: 2; YALI0E15400g having a nucleotide sequence of SEQ ID NO: 3; YALI0B01298g having a nucleotide sequence of SEQ ID NO: 4; and YALI0F23793g having a nucleotide sequence of SEQ ID NO: 5 with expression vectors each containing 16 UAS1B enhancers and a translational elongation factor (TEF) promoter. In this case, a transformation kit (Frozen-EZ Yeast Transformation II Kit (Zymo Research Corporation, California Irvine, USA)) may be used for the transformation. The expression vector consisting of the nucleotide sequence of SEQ ID NO: 9 is an exemplary expression plasmid comprising 16 UAS1B enhancers and a translational elongation factor (TEF) promoter in YALI0E15400g among the five aldehyde dehydrogenase genes. Alternatively, in one embodiment, the transformation may be performed through gene insertion using, for example, CRISPR/Cas9. In one embodiment, the order of the inserted genes is not limited.

In one embodiment, the expression vector may comprise, but is not limited to, genomic DNA or a plasmid. In one embodiment, each aldehyde dehydrogenase gene introduced into the expression vector may be inserted into genomic DNA or expressed in the form of a plasmid.

One embodiment of the present disclosure may provide a method for producing yeast oil comprising culturing the above-described furfural-resistant strain in a medium containing furfural.

In addition, one embodiment of the present disclosure may provide a method for neutralizing furfural, comprising converting furfural into 2-furoic acid by culturing the said furfural-resistant strain in a medium containing furfural. In this case, the method for neutralizing furfural refers to a method for converting furfural into 2-furoic acid, which is a relatively less toxic substance, in the cells of the strain.

According to one embodiment, the furfural-resistant strain converts furfural into 2-furoic acid, so that even in a medium containing furfural, the growth of the strain does not decrease, and high yeast oil production performance can be exhibited. For example, the method may comprise culturing the strain in a medium containing furfural at a concentration of 0.2 g/L or more, 0.3 g/L or more, 0.4 g/L or more, 0.5 g/L or more, 0.6 g/L or more, 0.7 g/L or more, 0.8 g/L or more, 0.9 g/L or more, 1 g/L or more, 1.2 g/L or more, 1.5 g/L or more, 1.7 g/L or more, or 1.9 g/L or more. In one embodiment, the concentration of furfural in the medium may be 2 g/L or less, but is not limited thereto.

Any culture method known in the art is applicable to the “culture” in the present disclosure without limitation. In one embodiment, the culture of the yeast strain may be any one selected from the group consisting of shaking culture, stationary culture, batch culture, fed-batch culture, and continuous culture. The shaking culture refers to a method of culturing a culture inoculated with a microorganism while shaking the culture. The stationary culture refers to a culture method in which a liquid culture inoculated with a microorganism is allowed to stand for culturing, without shaking. The batch culture refers to a culture method in which culture is performed with the volume of the culture fixed, without addition of a new culture from the outside. An apparatus capable of realizing this culture method is referred to as a batch reactor. The fed-batch culture is an opposite term of batch culture, in which all of the raw materials are added into a culture tank from the beginning and cultured, and it refers to a culture method in which a small amount of elements are first added, followed by repeated addition of a small amount of raw materials during culturing. An apparatus capable of realizing this culture method is referred to as a fed-batch reactor. The continuous culture refers to a culture method in which a new nutrient medium is continuously supplied and at the same time a culture containing cells and products is continuously removed. An apparatus capable of realizing this culture method is referred to as a continuous reactor. Specifically, the culture may be carried out in a batch reactor, a continuous reactor, or a fed-batch reactor.

In one embodiment, the culture may be performed in a common medium containing at least one selected from the group consisting of a suitable carbon source, nitrogen source, amino acid, vitamin, etc., under the culture conditions of yeast strains according to an appropriate method with adjustment of the temperature, pH, etc. Examples of carbon sources that may be used comprise sugars and carbohydrates such as glucose, xylose, sucrose, lactose, fructose, maltose, starch, and cellulose, oils and fats such as soybean oil, sunflower oil, castor oil, and coconut oil, fatty acids such as palmitic acid, stearic acid and linoleic acid, alcohols such as glycerol and ethanol, organic acids such as acetic acid, and volatile fatty acids (VFA) such as acetic acid, butyric acid, isobutyric acid, propionic acid, valeric acid, isovaleric acid, and caproic acid. These materials may be used alone or as a mixture. Specifically, the medium may contain at least one selected from the group consisting of glucose, xylose, arabinose, mannose, galactose, cellulose, hemicellulose, lignin, and lignocellulose as a carbon source. Examples of nitrogen sources that may be used comprise inorganic nitrogen sources such as ammonia, ammonium sulfate, ammonium chloride, ammonium acetate, ammonium phosphate, ammonium carbonate, and ammonium nitrate; and organic nitrogen sources, such as amino acids, peptones, glutamic acid, methionine and glutamine, NZ-amines, meat extract, yeast extract, malt extract, corn steep liquor, casein hydrolysate, fish or its decomposition products, and defatted soybean cake or its decomposition products. These nitrogen sources may be used alone or as a mixture. The medium may contain, as phosphorus sources, potassium phosphate monobasic, potassium phosphate dibasic and corresponding sodium-containing salts. Also, examples of inorganic compounds that may be used comprise sodium chloride, calcium chloride, iron chloride, magnesium sulfate, iron sulfate, manganese sulfate and calcium carbonate. Finally, in addition to the above materials, materials such as amino acids and vitamins may be used. In one embodiment, suitable precursors may be used in the culture medium. The above-mentioned raw materials may be added to the culture during culturing in a batch, a fed-batch, or a continuous manner by an appropriate method, although not particularly limited thereto. The pH of the culture may be adjusted by using an appropriate concentration of basic compounds, such as sodium hydroxide, potassium hydroxide, and ammonia or acidic compounds such as phosphoric acid or sulfuric acid in an appropriate amount and an appropriate manner.

In one embodiment, in the culturing step, the culturing temperature may be 22 to 37° C. Specifically, the culturing temperature may be 22° C. or more, 23° C. or more, 24° C. or more, 25° C. or more, 26° C. or more, 27° C. or more, 28° C. or more, 29° C. or more, 30° C. or more, 31° C. or more, 32° C. or more, 33° C. or more, 34° C. or more, or 35° C. or more, or the culturing temperature may be 37° C. or less, 36° C. or less, 35° C. or less, 34° C. or less, 33° C. or less, 32° C. or less, 31° C. or less, 30° C. or less, 29° C. or less, 28° C. or less, 27° C. or less, 26° C. or less, or 25° C. or less.

In one embodiment, the culturing time in the culturing step may be 24 to 200 hours. Specifically, the culturing time may be 24 hours or more, 36 hours or more, 48 hours or more, 60 hours or more, 78 hours or more, or 90 hours or more, or the culturing time may be 200 hours or less, 150 hours or less, 120 hours or less, 96 hours or less, 78 hours or less, 60 hours or less, 48 hours or less, or 36 hours or less. Specifically, the culturing step may comprise culturing the furfural-resistant strain in a medium at 24 to 32° C. for 24 to 200 hours.

In one embodiment, the medium may contain lignocellulosic biomass, and the method for producing yeast oil or the method for neutralizing furfural may further comprise pre-treating the lignocellulosic biomass prior to culturing the furfural-resistant strain in the medium. For example, the method may comprise strong-acid hydrolysate, weak-acid hydrolysate, organic solvent pretreatment, and the like.

In one embodiment, the lipids produced by the method for producing yeast oil, that is, yeast oil may be used as a raw material in various industrial fields such as bio-aircraft fuel and cosmetic materials.

Hereinafter, the present invention will be described in more detail through examples. However, it will be apparent to a person skilled in the art that the following examples are provided for illustrative purposes only to facilitate understanding of the present invention, and the scope of the present invention is not limited thereto.

### Reference Example 1

Confirmation of the degree of growth inhibition according to furfural in a parent strain Prior to the production of furfural-resistant strains, Yarrowia lipolytica PO1f strain having accession number ATCC MYA-2613, which are wild-type Yarrowia lipolytica, was selected as a parent strain, and the degree of growth inhibition and the degree of glucose consumption according to the concentration of furfural in the wild-type Yarrowia lipolytica strain, and the concentrations of furfural and furoic acid in the culture medium according to the cell culture time were confirmed.

Specifically, as the culture medium, a minimal medium (YSC medium (Yeast Synthetic Complete medium)) containing 6.7 g/L yeast nitrogen base (YNB), 20 g/L glucose as a carbon source and supplemented with complete supplement mixture (CSM) or CSM-Leu or CSM-Leu-Ura (MP Biomedicals, Solon, OH, USA) used. Furfural was added to the medium at concentrations of 0.1 g/L, 0.2 g/L, 0.3 g/L, 0.4 g/L or 0.5 g/L, respectively, and the growth rate was compared with that when furfural was not added. The culture was inoculated with the wild-type Yarrowia lipolytica strain with an initial OD₆₀₀ value of 0.2 in 20 mL of the culture medium in a 100 mL Erlenmeyer flask, and then proceeded with shaking culture at 28° C. at 200 rpm. In this case, the wild-type strain is Yarrowia lipolytica PO1f strain with accession number ATCC MYA-2613. The “OD₆₀₀” refers to the absorbance or optical density at a wavelength of 600 nm. It is possible to measure the density or concentration of microorganisms or cells in a sample by using the measured OD₆₀₀.

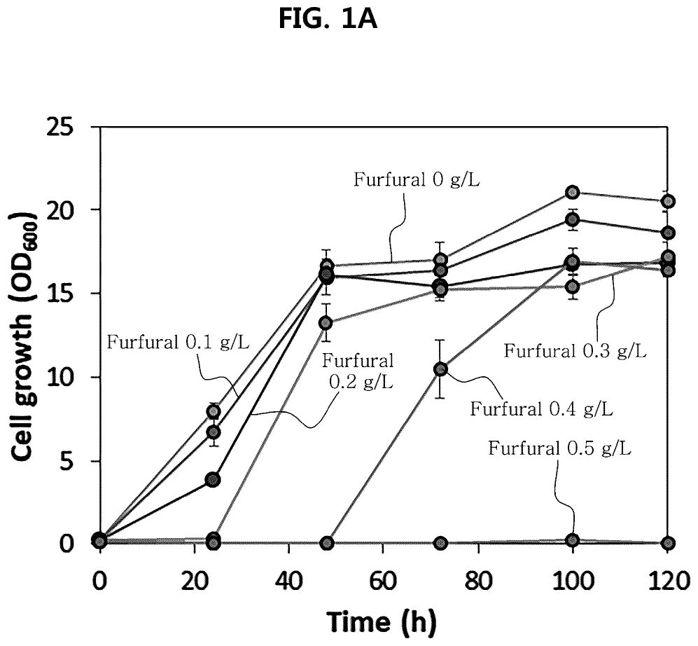

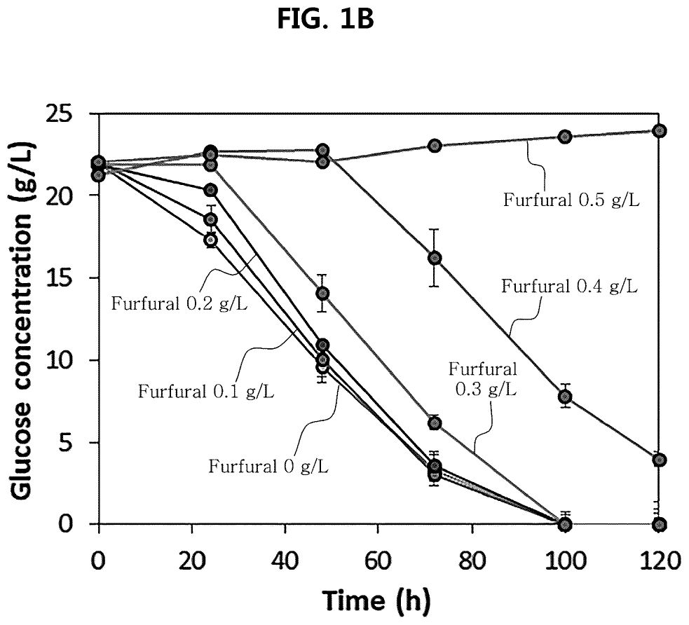

As a result of the culture, as shown in FIGS. 1A and 1B, as the furfural concentration increased, it was confirmed that the growth of the wild-type Yarrowia lipolytica strain was clearly inhibited and the rate of glucose consumption was reduced. In particular, it was confirmed that growth was delayed in the initial growth under the condition of 0.2 g/L or more of furfural, and no growth was observed under the condition of 0.5 g/L of furfural.

### Confirmation of Furfural Resistance of Wild-Type Yarrowia Lipolytica Strain by Alcohol Dehydrogenase or Aldehyde Reductase

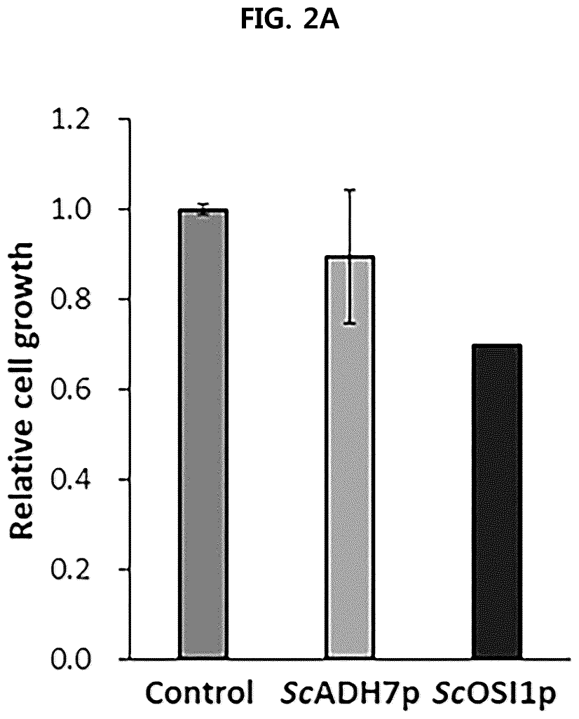

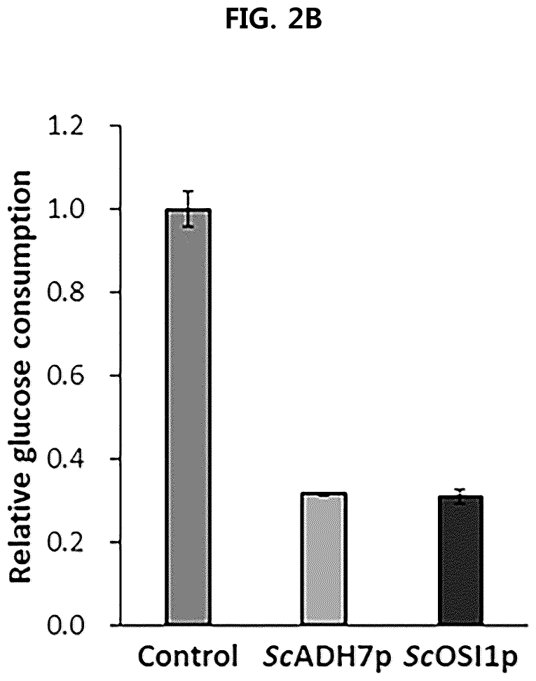

Since the growth inhibition of wild-type Yarrowia lipolytica strain caused by furfural was confirmed, as a method for neutralizing growth inhibition, it was confirmed whether the expression of alcohol dehydrogenase and aldehyde reductase, which are popularly used in common yeast strains, also worked in Yarrowia lipolytica strain. Specifically, an experiment was performed in which the strain transformed with two alcohol dehydrogenases (ScADH7p) and aldehyde reductase (ScOSI1p) whose efficacy was previously verified in Saccharomyces cerevisiae was cultured in a medium supplemented with 0.4 g/L furfural. The culture condition was the same as in Reference Example 1. That is, the initial OD₆₀₀ value was 0.2, and the minimum medium contained 20 g/L of glucose as a carbon source, and the culture was carried out for up to 170 hours by shaking culture at 200 rpm at 28° C. in 20 mL of the culture medium in a 100 mL Ellenmeyer flask. As a result, as shown in FIGS. 2A and 2B, it was confirmed that, unlike other strains, alcohol dehydrogenase or aldehyde reductase had no effect or had an adverse effect on furfural neutralization in Yarrowia lipolytica. Therefore, it was confirmed that it was difficult to enhance the furfural resistance of Yarrowia lipolytica by conventional overexpression of alcohol conversion enzymes (alcohol dehydrogenase and aldehyde reductase).

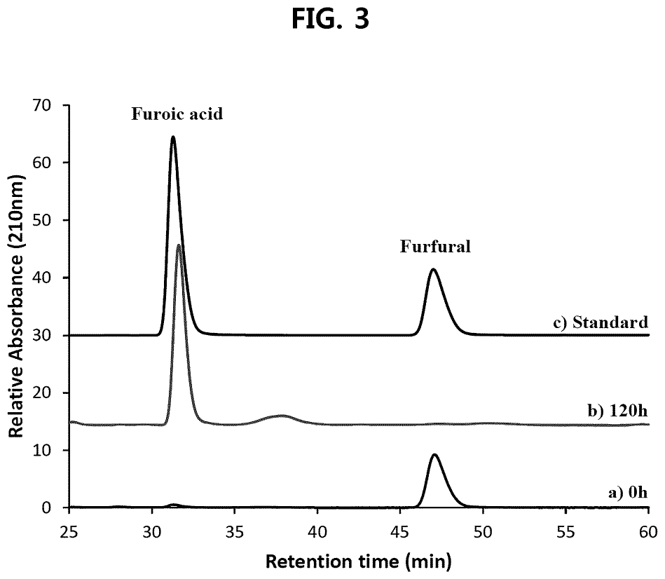

In addition, in the culture using the culture medium to which 0.4 g/L of furfural was added, liquid chromatography (HPLC) was performed to determine the change in the concentration of furfural and furoic acid according to the growth of wild-type Yarrowia lipolytica strain in the culture medium at the beginning and end of the culture. In this case, the sample used for analysis was prepared by centrifuging the strain in the culture medium at 13,000 rpm for 1 minute to separate supernatant from the culture medium, and then treating the supernatant with a 0.2 m filter and diluting the supernatant by 1/10. As a result of the analysis, unlike the fact that only furfural was present in the first time period (0 h) as shown in FIG. 3, no furfural was present and only the presence of furoic acid could be observed at 120 hours (120 h), the time period when the culture was finished. Therefore, it can be confirmed that furfural added to the medium is converted into furoic acid by the action of cells.

### Example 1

### Target Gene Screening and Identification for Discovery of Furfural Neutralization-Related Genes

The present inventors focused on Reference Example 1 in which the growth of the wild-type Yarrowia lipolytica strain was inhibited by furfural added to the medium, but the strain could convert furfural to furoic acid, and thus, performed experiments below to screen the genes related to furfural neutralization as a gene for enhancing resistance to furfural. Specifically, based on EcAldH (PuuC, SEQ ID NO: 1), an aldehyde dehydrogenase of E. coli, a representative strain, homology search was performed to find a gene similar to EcAldH in the wild-type Yarrowia lipolytica using BlastP (https://blast.ncbi.nlm.nih.gov/Blast.cgi).

As a result, a total of five aldehyde dehydrogenases unique to Yarrowia lipolytica, YALI0F04444g (YER073w-like aldehyde Dehydrogenase, SEQ ID NO: 6), YALI0E00264g (YOR374w-like aldehyde Dehydrogenase, SEQ ID NO: 7), YALI0D07642g (YMR170c-like aldehyde Dehydrogenase, SEQ ID NO: 8), YALI0E15400g (Fatty aldehyde dehydrogenase 2, SEQ ID NO: 3), and YALI0B01298g (Fatty aldehyde dehydrogenase 3, SEQ ID NO: 4) were selected.

In order to confirm the furfural neutralization effect of the target genes selected in the homology search, an experiment was performed in which transformant strains expressing each of the target genes were cultured in a medium supplemented with 0.4 g/L of furfural.

Specifically, the target genes YALI0F04444g, YALI0E00264g, YALI0D07642g, YALI0E15400g, and YALI0B01298g were expressed by using a Yarrowia lipolytica expression vector, pMCS (SEQ ID NO: 11) containing 16 UAS1B enhancers and translational elongation factor (TEF) promoters, respectively. The wild-type Yarrowia lipolytica strain was transformed using a transformation kit (Frozen-EZ Yeast Transformation II Kit; Zymo Research Corporation, California Irvine, USA).

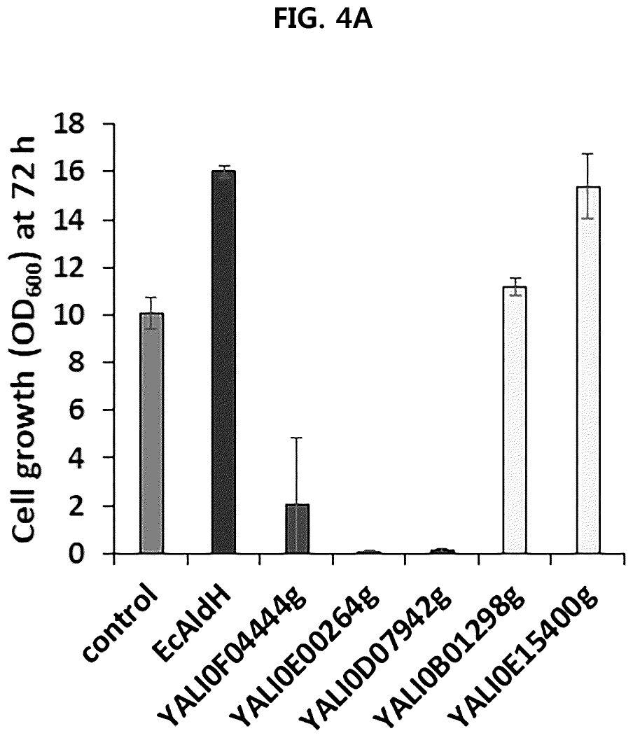

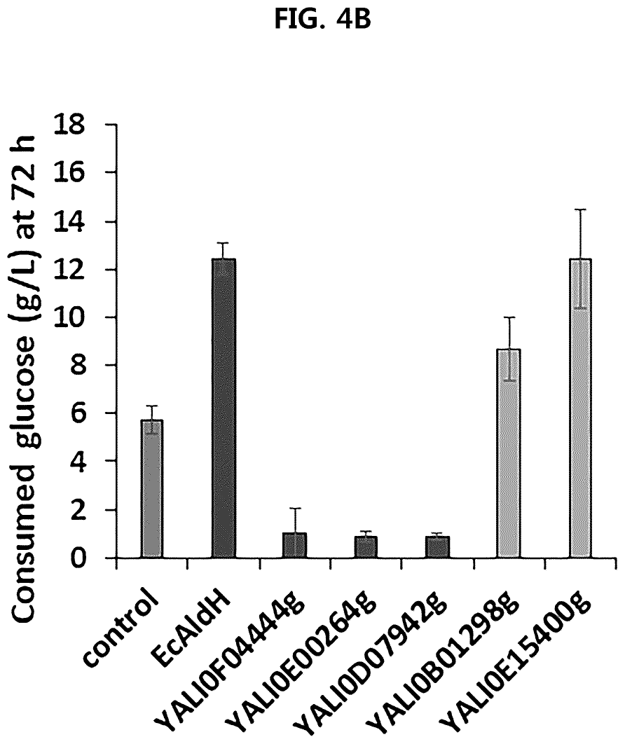

The recombinant strain obtained through the transformation was inoculated with an initial OD₆₀₀ value of 0.2 and cultured. As for the culture conditions, 20 g/L of glucose was used as the carbon source in the minimal medium in the same manner as in Reference Example 1, and under the condition of adding 0.4 g/L of furfural, the culture was carried out by shaking culture at 200 rpm at 28° C. in 20 mL of the culture medium in a 100 mL Erlenmeyer flask. Then, the degree of cell growth (OD₆₀₀) and glucose consumption rate at the time of culture at 72 hours were compared and shown in FIGS. 4A and 4B.

As a result, it was confirmed that YALI0F04444g, YALI0E00264g, and YALI0D07942g, which were selected as the target genes, had a lower growth rate than that in the wild-type strain, and the amount of glucose consumed was also very small. On the other hand, in the case of YALI0B01298g and YALI0E15400g, compared to the wild-type strain, the total amount of biomass increased by 10.9% and 52.9% based on OD₆₀₀, and the amount of glucose consumed increased by 52% and 118%. Summarizing the results in FIGS. 4A and 4B, it was confirmed that not all aldehyde dehydrogenases in Yarrowia lipolytica were simply effective.

### Confirmation of the Effect of Target Genes in Furfural-Added Medium

Based on YALI0E15400g, which showed the best effect in the above experiment, homology search was performed again within the entire nucleotide sequence of Yarrowia lipolytica, and new target genes YALIA17875g (SEQ ID NO: 2) and YALI0F23793g (SEQ ID NO: 5) were selected.

In the same way as the method used in the above experiment, the genes were expressed using pMCS (SEQ ID NO: 11), a Yarrowia lipolytica expression vector containing 16 UAS1B enhancers and a translational elongation factor (TEF) promoter, so that Yarrowia lipolytica transformant strains in which aldehyde dehydrogenase (EcAldH, SEQ ID NO: 1), YALIA17875g (SEQ ID NO: 2), YALI0E15400g (SEQ ID NO: 3), YALI0B01298g (SEQ ID NO: 4) or YALI0F23793g (SEQ ID NO: 5) according to an embodiment of the present disclosure were overexpressed were prepared respectively.

For example, the Yarrowia lipolytica transformant strain overexpressing YALI0E15400g (SEQ ID NO: 3) was transformed by inserting the YALI0E15400g expression plasmid of SEQ ID NO: 9 using the Yarrowia lipolytica PO1f strain (accession number ATCC MYA-2613), a wild-type Yarrowia lipolytica strain. Specifically, 10 mL of a wild-type strain that reached an OD₆₀₀ of 1.0 in YPD (yeast extract peptone dextrose agar) medium containing 20 g/L glucose was separated by centrifugation at 500 g for 4 minutes, and the transformation kit (Frozen-EZ Yeast Transformation II Kit; Zymo Research Corporation, California Irvine, USA) was used to prepare a recombinant strain through transformation.

Culture conditions are also the same as in Reference Example 1 with an initial OD₆₀₀ value of 0.2 and 20 g/L of glucose as carbon sources and 0.4 g/L of furfural added to the minimum medium. The culture was carried out by shaking culture at 200 rpm at 28° C. in 20 mL of the culture medium in a 100 mL Ellenmeyer flask.

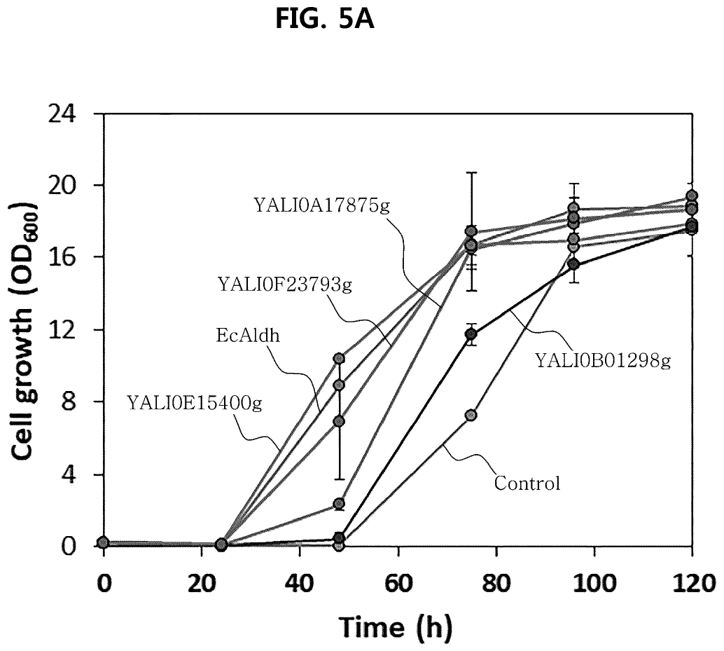

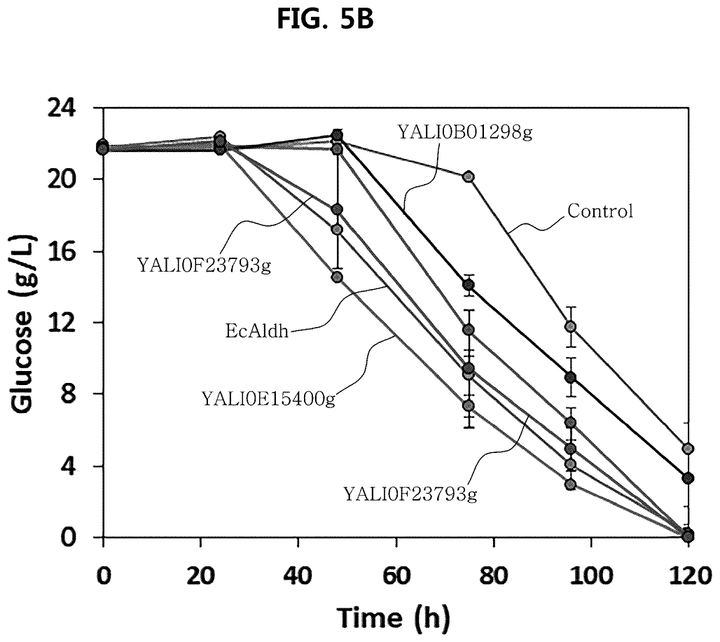

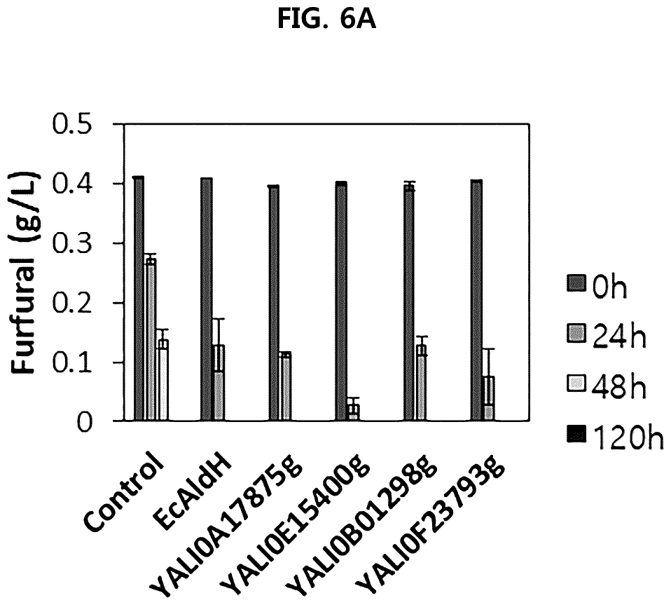

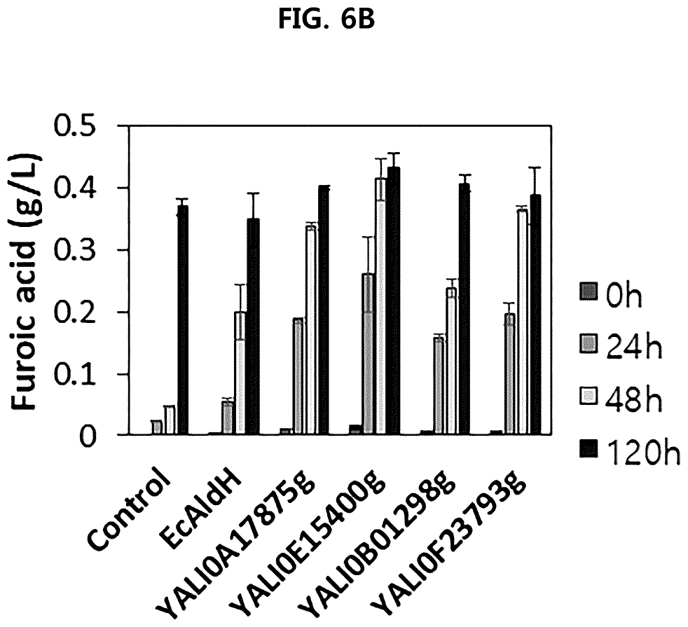

As a result of the analysis, as shown in FIGS. 5A and 5B, the all transformant strains with overexpression of aldehyde dehydrogenase (EcAldH, SEQ ID NO: 1), YALIA17875g (SEQ ID NO: 2), YALI0E15400g (SEQ ID NO: 3), YALI0B01298g (SEQ ID NO: 4) or YALI0F23793g (SEQ ID NO: 5) according to an embodiment of the present disclosure showed high cell growth compared to the negative control (the wild-type Yarrowia lipolytica strain) and high glucose consumption of 53.8% under the 0.4 g/L condition. In addition, as shown in FIGS. 6A and 6B, it was confirmed that in the transformant strain with overexpression of aldehyde dehydrogenase (EcAldH, SEQ ID NO: 1), YALIA17875g (SEQ ID NO: 2), YALI0E15400g (SEQ ID NO: 3), YALI0B01298g (SEQ ID NO: 4) or YALI0F23793g (SEQ ID NO: 5) according to an embodiment of the present disclosure, furfural was quickly reduced and converted to furoic acid compared to the wild-type Yarrowia lipolytica strain. Among them, it was confirmed that the transformant strain overexpressing YALI0E15400g showed 16.7% higher growth and 53.8% higher glucose consumption compared to the growth of the transformant strain overexpressing E. coli aldehyde dehydrogenase (EcAldH); showed the conversion rate 11 times faster than that of the wild-type strain and 4.61 times faster than that of the transformant strain overexpressing E. coli aldehyde dehydrogenase at 24 hours of incubation time.

Therefore, the furfural resistance of the Yarrowia lipolytica transformant strain overexpressing one of aldehyde dehydrogenase (EcAldH, SEQ ID NO: 1), YALIA17875g (SEQ ID NO: 2), YALI0E15400g (SEQ ID NO: 3), YALI0B01298g (SEQ ID NO: 4) and YALI0F23793g (SEQ ID NO: 5) according to an embodiment of the present disclosure can be confirmed through the furfural neutralization ability.

### Example 2

The production performance of yeast oil was analyzed using the strain transformed with YALI0E15400g having the best furfural neutralization ability in the presence of furfural in Example 1.

First, Yarrowia lipolytica PO1f strain (accession number ATCC MYA-2613), a wild-type Yarrowia lipolytica strain, was transformed by inserting the YALI0E15400g expression plasmid of SEQ ID NO: 9. Specifically, 10 mL of wild-type strain that reached an OD₆₀₀ of 1.0 in YPD (Yeast Extract Peptone Dextrose) medium containing 20 g/L glucose were separated by centrifugation at 500 g for 4 minutes, and the transformation kit (Frozen-EZ Yeast Transformation II Kit; Zymo Research Corporation, California Irvine, USA) was used for transformation to prepare the recombinant strain (w/o DGA1; Yarrowia lipolytica WF). The YPD medium was prepared directly, in this case, yeast extract was purchased from Becton Dickinson & Company, New Jersey (USA), peptone was purchased from Becton Dickinson & Company (USA), and dextrose (glucose) was purchased from Junsei Chemical, Tokyo (Japan).

In another embodiment of the present disclosure, a Yarrowia lipolytica transformant strain overexpressing YALI0E15400g was prepared in a recombinant strain (po1f DGA1) with an enhanced yeast oil production pathway. Specifically, by transforming the wild-type Yarrowia lipolytica strain with the YALI0E15400g expression plasmid of SEQ ID NO: 9 and the DGA1 expression plasmid (SEQ ID NO: 10) in the same manner as in the recombinant strain (w/o DGA1; Yarrowia lipolytica WF), the recombinant strain (w/DGA1; Yarrowia lipolytica LF) was prepared.

The recombinant strain (Yarrowia lipolytica WF) and the recombinant strain (Yarrowia lipolytica LF) were deposited with accession numbers KCTC15376BP and KCTC15377BP on Mar. 17, 2023, respectively.

The culture conditions of the two strains were the same as in Reference Example 1, with an initial OD₆₀₀ value of 0.2, and 20 g/L of glucose as a carbon source and 0.4 g/L of furfural added to the minimum medium. The culture was carried out by shaking culture at 200 rpm at 28° C. in 20 mL of culture medium in a 100 mL Erlenmeyer flask. For the Nile Red staining method, 100 L of the culture medium was centrifuged at 10,000 g for 1 minute to isolate the matured strain, and then dissolved again in 500 μL of pH 6.8 PBS buffer solution, then 1 μL of 1 mM Nile Red solution dissolved in DMSO was added and dark reaction was performed at 30° C. for 15 minutes. The sample stained with Nile Red was measured at 535 nm and 580 nm of measurement wavelength after an additional washing process.

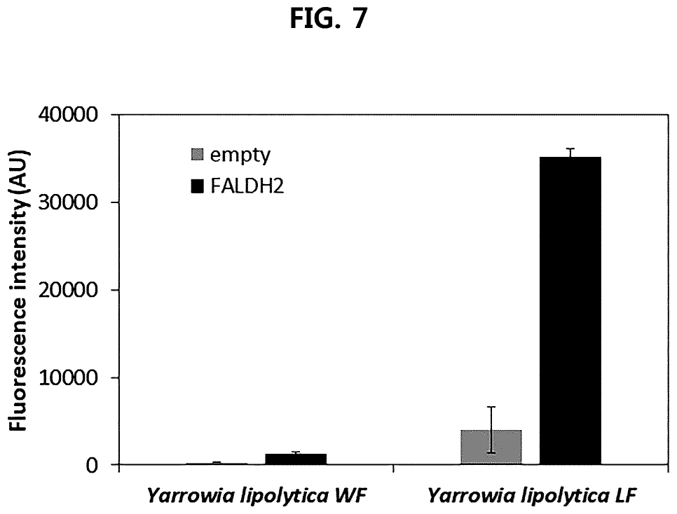

As a result, as shown in FIG. 7, the two Yarrowia lipolytica transformant strains overexpressing YALI0E15400g according to an embodiment of the present disclosure (shown as FALDH2 in FIG. 7) were evaluated for yeast oil production performance by Nile Red staining method. It was confirmed that the strains transformed with YALI0E15400g showed the yeast oil production performance that was 4 times and 8.8 times higher than that of the non-transformed strain (shown as empty in FIG. 7), respectively.

Therefore, it can be confirmed that the furfural resistance of the Yarrowia lipolytica strain can be enhanced through the expression of aldehyde dehydrogenase according to an embodiment of the present disclosure, and the yeast oil production performance can be significantly improved.

The present disclosure may provide the following embodiments as an example.

First embodiment may provide a furfural-resistant strain comprising a gene encoding an aldehyde dehydrogenase (EcAldH) gene, wherein the aldehyde dehydrogenase gene comprises at least one gene sequence selected from the group consisting of EcAldH (PuuC) having a nucleotide sequence of SEQ ID NO: 1; YALI0A17875g having a nucleotide sequence of SEQ ID NO: 2; YALI0E15400g having a nucleotide sequence of SEQ ID NO: 3; YALI0B01298g having a nucleotide sequence of SEQ ID NO: 4; and YALI0F23793g having a nucleotide sequence of SEQ ID NO: 5.

The second embodiment may provide the furfural-resistant strain according to the second embodiment, wherein the furfural-resistant strain is a transformant strain to comprise the gene encoding the aldehyde dehydrogenase gene using Yarrowia lipolytica as a parent strain.

The third embodiment may provide the furfural-resistant strain according to the second embodiment, wherein the parent strain is a wild-type Yarrowia lipolytica strain.

The fourth embodiment may provide the furfural-resistant strain according to the second or third embodiment, wherein the parent strain is a strain transformed with a DGA1 expression vector consisting of a nucleotide sequence of SEQ ID NO: 10 in the wild-type Yarrowia lipolytica strain.

The fifth embodiment may provide the furfural-resistant strain according to any one of the first to fourth embodiments, wherein the furfural-resistant strain is Yarrowia lipolytica WF deposited under accession number KCTC15376BP.

The sixth embodiment may provide the furfural-resistant strain according to any one of the first to fourth embodiments, wherein the furfural-resistant strain is Yarrowia lipolytica LF deposited under accession number KCTC15377BP.

The seventh embodiment may provide a method for producing yeast oil comprising culturing the furfural-resistant strain of any one of the first to sixth embodiments in a medium containing furfural.

The eight embodiment may provide the method according to the seventh embodiment, wherein the medium contains the furfural at a concentration of 0.2 g/L or more.

The ninth embodiment may provide the method according to the seventh or eighth embodiment, wherein the medium contains at least one selected from the group consisting of glucose, xylose, arabinose, mannose, galactose, cellulose, hemicellulose, lignin, and lignocellulose as a carbon source.

The tenth embodiment may provide the method according to any one of the seventh to ninth embodiments, wherein the culture is carried out for 24 to 200 hours at 22 to 37° C.

The eleventh embodiment may provide a method for neutralizing furfural comprising converting furfural into 2-furoic acid by culturing the furfural-resistant strain according to any one of the first to sixth embodiments in a medium containing furfural.

The twelfth embodiment may provide the method according to the eleventh embodiment, wherein the conversion of the furfural to the 2-furoic acid is promoted through an expression of the aldehyde dehydrogenase.

The thirteenth embodiment may provide the method according to the eleventh or twelfth embodiment, wherein the medium contains the furfural at a concentration of 0.2 g/L or more.

The fourteenth embodiment may provide the method according to any one of the eleventh to thirteenth embodiments, wherein the medium contains at least one selected from the group consisting of glucose, xylose, arabinose, mannose, galactose, cellulose, hemicellulose, lignin, and lignocellulose as a carbon source.

The fifteenth embodiment may provide the method according to any one of the eleventh to fourteenth embodiments, wherein the culture is carried out for 24 to 200 hours at 22 to 37° C.

### [Accession Number]

Depository Institution: Korean Collection for Type Cultures (KCTC) of Korea Research Institute of Biotechnology and Bioscience
Accession number: KCTC15376BP
Deposit date: 20230317
Depository Institution: Korean Collection for Type Cultures (KCTC) of Korea Research Institute of Biotechnology and Bioscience
Accession number: KCTC15377BP
Deposit date: 20230317

What is claimed is:

1. A furfural-resistant strain comprising a gene encoding an aldehyde dehydrogenase (EcAldH) gene,
wherein the aldehyde dehydrogenase gene comprises at least one gene sequence selected from the group consisting of EcAldH (PuuC) having a nucleotide sequence of SEQ ID NO: 1; YALI0A17875g having a nucleotide sequence of SEQ ID NO: 2; YALI0E15400g having a nucleotide sequence of SEQ ID NO: 3; YALI0B01298g having a nucleotide sequence of SEQ ID NO: 4; and YALI0F23793g having a nucleotide sequence of SEQ ID NO: 5.

2. The furfural-resistant strain of claim 1, wherein the furfural-resistant strain is a transformant strain to comprise the gene encoding the aldehyde dehydrogenase gene using Yarrowia lipolytica as a parent strain.

3. The furfural-resistant strain of claim 2, wherein the parent strain is a wild-type Yarrowia lipolytica strain.

4. The furfural-resistant strain of claim 2, wherein the parent strain is a strain transformed with a DGA1 expression vector consisting of a nucleotide sequence of SEQ ID NO: 10 in the wild-type Yarrowia lipolytica strain.

5. The furfural-resistant strain of claim 1, wherein the furfural-resistant strain is Yarrowia lipolytica WF deposited under accession number KCTC15376BP.

6. The furfural-resistant strain of claim 1, wherein the furfural-resistant strain is Yarrowia lipolytica LF deposited under accession number KCTC15377BP.


SEQUENCE LISTING (11 sequences)

SEQ ID NO: 1 - DNA, 1488 bp, synthetic construct
  source 1..1488: mol_type = other DNA
atgaattttcatcatctggcttactggcaggataaagcgttaagtctcgccattgaaaaccgcttatttattaacggtgaatatactgctgcggcggaaaatgaaacctttgaaaccgttgatccggtcacccaggcaccgctggcgaaaattgcccgcggcaagagcgtcgatatcgaccgtgcgatgagcgcagcacgcggcgtatttgaacgcggcgactggtcactctcttctccggctaaacgtaaagcggtactgaataaactcgccgatttaatggaagcccacgccgaagagctggcactgctggaaactctcgacaccggcaaaccgattcgtcacagtctgcgtgatgatattcccggcgcggcgcgcgccattcgctggtacgccgaagcgatcgacaaagtgtatggcgaagtggcgaccaccagtagccatgagctggcgatgatcgtgcgtgaaccggtcggcgtgattgccgccatcgtgccgtggaacttcccgctgttgctgacttgctggaaactcggcccggcgctggcggcgggaaacagcgtgattctaaaaccgtctgaaaaatcaccgctcagtgcgattcgtctcgcggggctggcgaaagaagcaggcttgccggatggtgtgttgaacgtggtgacgggttttggtcatgaagccgggcaggcgctgtcgcgtcataacgatatcgacgccattgcctttaccggttcaacccgtaccgggaaacagctgctgaaagatgcgggcgacagcaacatgaaacgcgtctggctggaagcgggcggcaaaagcgccaacatcgttttcgctgactgcccggatttgcaacaggcggcaagcgccaccgcagcaggcattttctacaaccagggacaggtgtgcatcgccggaacgcgcctgttgctggaagagagcatcgccgatgaattcttagccctgttaaaacagcaggcgcaaaactggcagccgggccatccacttgatcccgcaaccaccatgggcaccttaatcgactgcgcccacgccgactcggtccatagctttattcgggaaggcgaaagcaaagggcaactgttgttggatggccgtaacgccgggctggctgccgccatcggcccgaccatctttgtggatgtggacccgaatgcgtccttaagtcgcgaagagattttcggtccggtgctggtggtcacgcgtttcacatcagaagaacaggcgctacagcttgccaacgacagccagtacggccttggcgcggcggtatggacgcgcgacctctcccgcgcgcaccgcatgagccgacgcctgaaagccggttccgtcttcgtcaataactacaacgacggcgatatgaccgtgccgtttggcggctataagcagagcggcaacggtcgcgacaaatccctgcatgcccttgaaaaattcactgaactgaaaaccatctggataagcctggaggcctga

SEQ ID NO: 2 - DNA, 1602 bp, synthetic construct
  source 1..1602: mol_type = other DNA
atgtcctgggaaacaatcactcctcctacgccaatcgatacgtttgacagcaacttgcaacgtcttcgagactctttcgagaccggcaagctcgactctgtcgactaccgtctcgagcagctgcgaaccctgtggttcaagttctacgacaacctcgacaacatctacgaggcggtcaccaaggatctccatcgacccaggttcgaaaccgagctcaccgaggtactgtttgttcgagacgagttctccaccgtcatcaagaacctgcgaaagtgggtcaaggaagaaaaggtggagaaccccggaggccccttccagtttgccaacccccgaatccgacccgttcctctgggagtggtgctggtcatcactccctggaactaccccgtcatgctcaacatctcacctgtgattgccgccattgctgccggctgtcccatcgtgctcaagatgtccgagctgtctccccacacttccgctgttcttggccgaatcttcaaggaggccctggaccccggtatcatccaggttgtttacggaggtgtccccgagaccaccgcccttcttacccagcattgggacaagatcatgtacaccggaaacggagccgttggtcgaatcatcgcccaggccgcggtcaagaacctgactcctctagctcttgagcttggtggcaagtcacccgtgttcatcacttccaactgcaagagcgttatgacggccgctcggcgaatcgtgtggggcaagtttgtcaacgccggccagatctgtgtcgctccagactacattctggttgctcccgaaaaggaggccgagctcgtcgcttgtatcaaggaggtgctccaagaacgatacggctccaagagagacgcccaccaccccgatctgtcccatatcatttccaagccccattggaagcgtattcacaacatgatcgcccagaccaagggagacatccaggtgggtggactcgagaacgccgacgaagaccaaaagttcatccagcccacaatcgtctccaacgttccagatgacgacattctcatgcaggacgagattttcggacccatcatccccatcatcaagccccgaaccctcggccagcaggttgattacgtcacaagaaaccatgacacccccctggccatgtacatcttctctgacgaccccaaggaggtggactggctacagacccgaatccgagctggttctgtaaacatcaacgaggtcattgagcaggtcggactggcctctctgcctctcagtggagttggagcttccggaaccggagcataccatggaaaattctccttcgatgtcttcacccacaagcaggccgttatgggacagcccacctggcccttctttgaatacctcatgtattaccggtaccctccttactccgagtacaagatgaaggtgctccgaaccctgttcccaccggttctgattcctcgaaccggccgacccgacgctactgttcttcagcgagttctcggcaacaagctgctttggatcattattgccgcccttgttgcgtacgccaaacgaaatgagctgctcatcaccattgctcagattatgtcggtgtttattaagtag

SEQ ID NO: 3 - DNA, 1566 bp, synthetic construct
  source 1..1566: mol_type = other DNA
atgtcagagttcgattgggagtcaattttgccggcaacaccactaggtgagatcgagaaggatattcaaaccctacgacagggcttcaggtccggaaagacgctggatttgaacttcaggcttgaccagattcgtaagcttttctatgctctctatgataatgtcgatgcgatcaaagaagcaattcataaggatctcggacgtccggtcttcgagactgaactttgcgagatctcctttcagtggggtgaattcaataatgtcgtttctaacttgaagaaatgggcagctgatgagacggtgaagggaaccaccattcaatacactctcacccggccaaagattagaaagcgtccacttggtaccgtccttatcatatctccttggaactacccatttgttctgaccatctctcccctgcttgctgctctagcggcaggaaatacggtggccctaaagttctccgaaatgtgcccacatacatcgcttttgctgggaaagttgtgcacagaggcacttgataaagaaattttcaaggcatttcagggaggcgttccggtagtgtcggagattctcaagtacaagttcgacaaaatcatgtacactggaaatcatcgagttggcaagatcatcttggacgcagctaacaaatacctcacccccgttattttggagcttggaggcaaatcaccagtcttcgtgactaagaattgccaaaacgtatctcttgctgccaagcgtgctctgtggggtaaactggtcaacgctggacaaacatgcgttgcccccgattacatcatcgtcgagcctgaggtcgaacaggagtttatcaaagcttgccagtactgggttgagaagttctaccgaggtggagttgactctgatcataaggacttcactcatattgcaacacctggacattggagacgattgacatccatgcttgcccagacagagggaaatatcatcacaggcggaaattcggacgagaaatcacggtttcttgctcccacagttgttgcgaaagttcctgatggtgattctttgatgaatgatgagatctttggccctatcctgcccatcctgacagccagatccgttgacgaaggtattcgctatgttcatgagaatcacgacactcccctggccatgtatgtctttactgataatgcatcagaaggagagtatatccaatctcaaatcaactcaggtggcctgatattcaatgatagtcttgttcacgttggctgcgtgcaggcgccttttggtggtgtcggccaatccggctatgggtcttatcacggcgaagattccttcttggctttttcacacaggcagactttcatgaagcagccccatttcatcgaacgaccaatggcgatcagatatgccccctacactagtcgaaaacaaaaggctgtccagggtagtctagctgctccatcttttcctcgaacaggaaaggttgaccgctccctgttggagcggatatttggtaagctatggttctgggtgatcgttttagggctaggagcagccagtttgaagtcaggaattttcttatga

SEQ ID NO: 4 - DNA, 1590 bp, synthetic construct
  source 1..1590: mol_type = other DNA
atgactaccactgccacagagacccccacgacaaacgtgacccccaccacgtcactgcccaaggagaccgcctccccaggagggaccgcttctgtcaacacgtcattcgactgggagagcatctgcggcaagacgccgttggaggagatcgagtcggacatttcgcgtctcaaaaagaccttccgatcgggcaaaactctggatctggactaccgactcgaccagatccgaaacctggcgtatgcgatccgcgataacgaaaacaagatccgcgacgccatcaaggcggacctgaaacgacctgacttcgaaaccatggcggccgagttctcggtccagatgggcgaattcaactacgtggtcaaaaacctgccgaaatgggtcaaggacgaaaaagtcaagggaaccagcatggcgtactggaactcgtcgccaaagatccggaaacggcccctgggctccgtgcttgtcatcacgccctggaactacccactgattctggccgtgtcgcctgttctgggcgccattgccgcaggcaacaccgtggcgctgaaaatgtcagaaatgtcacccaacgcgtcaaaggtgattggcgacattatgacagctgccctggacccccagctctttcaatgcttcttcggaggagtccccgaaaccaccgagatcctcaaacacagatgggacaagatcatgtacaccggaaacggcaaagtgggccgaatcatctgtgaggctgccaacaagtacttgacacctgtggagctcgaactcggaggaaagtcgcctgttttcgtcaccaaacactgctccaacctggaaatggccgcccgccgaatcatctggggcaaattcgtcaacggaggacaaacctgcgtggctccagactacgttctggtgtgtcccgaggtccacgacaaatttgtggctgcctgtcaaaaggtgctggacaagttctaccctaacaactctgccgagtccgagatggcccatatcgccacccctctccattacgagcgtttgacgggcctgctcaattccacccgaggtaaggtcgttgctggaggcactttcaactcggccacccggttcattgctcctacgattgtcgacggagtggatgccaacgattctctgatgcagggagaactgtttggtcctcttctccccattgtcaaggccatgagcaccgaggctgcctgcaactttgtgcttgagcaccaccccacccccctggcagagtacatcttttcagataacaattctgagattgattacatccgagatcgagtgtcgtctggaggtctcgtgatcaacgacactctgatccacgtgggatgcgtacaggcgccctttggaggtgtcggagacagtggaaatggaggataccatggcaagcacactttcgatttgttcagccattctcagacggtcctcagacaacccggatgggtcgaaatgctgcagaagaaacggtatcctccgtacaacaagagcaacgagaagtttgtccggagaatggtggtccccagccctggttttccccgggagggtgacgtgagaggattttggtcgagactcttcaactag

SEQ ID NO: 5 - DNA, 1560 bp, synthetic construct
  source 1..1560: mol_type = other DNA
atgtctacctttgattgggaatccattgtgcctgccactcctctcgaccagattcctggcgacatccagcgactgcgaaagggcttccgatccggaaagaccctcgatctcaactaccgactggaccagattcgaaacttgcactacgtcctcagagacaatgtcgaggccatcaaggacgccgtgtacaaggatctcggccgacccaagcacgagactgacctgtgcgaggtgggtttcctgtggggcgagtttaacaacgtggttgccaacctcaagaagtgggccgccgacgaggacgtcaagaccaacctgcagtactccatctcctcccccaagatccgaaagcgacctcttggaaacgtgctcatcatctcgccctggaactacccctttatgctgaccgtgtctcctctcattggagctctggctgccggtaacactgtggctgtcaagttctccgaaatggccccccacacttccaaaattgttggcgacttgtgcaccaaggccctcgaccccgacgtcttccaggccatccagggaggtgtccccgtcgtcaccaagaccctcgagcagaagttcgacaagattatgtacactggtaaccacactgtcggtaagatcattgccactgccgccaacaagtacctgacacccgtcatcctcgagctcggaggtaagtcgcccgtttttgtcaccaagaactgcaagaacatcaagcttgccgctaagcgagccctgtggggtaaggtggtaaacgctggccagacctgtgtggctcccgactacgtgattgtcgagcccgaggtggagcaggagtttatcgacgcctgcaagtactggattaacgagttctacagtggtaagattgaccagtacaaccccgactttgccaagatcgccacccccaaccactggaaccgacttacctccatgttgagcaagtccaagggagagatcattactggaggtaacactgacgagaagactcgattcatcgctcctactgtcgtcgcaaaggtccccgacaatgattccctgatggaggacgagattttcggccctcttctgcccattctcactgcccgatccgtcgaggagggtatcaagtacgtgcacgagaaccacgacacccctcttgccatgtacgtcttcactgacaaggcctctgagggcgactacatccagtcccagatcaactctggtggccttatcttcaatgacactctgatccacgttggatgtgtccaggctccgtttggtggtgtcggcatgtccggttacggtgcttaccatggcgaggactccttcctggccttcacccaccgacaaacctacctcaaccagcccaagcttctggagcctcttcaggacgtgcgatacgccccctacaccaaaaccaagcgaagcatggtcaagaacctgctgctggtcggccccattttcccccgaaccggctccgtataccccaacgtgctgatccgaatcttccgaaagatttggttctgggtccttattgtcgccatcggagctgctggtgccaaggctctgctctag

SEQ ID NO: 6 - DNA, 1548 bp, synthetic construct
  source 1..1548: mol_type = other DNA
atgccatatatacggtgtctgggggactgtatcggtcccatcaaccaattgctcaacatgtccatctccatttcgctgcccaacggaaacaagtacgaacagcccacgggcattttcatcaacaacgagtggtccgaggcctccgacaagggtaccattcccgtctacaacccgtcgaccggcgacgaggtggtgcaggtggcggctgctactgctgaggacgtggatcgggcagtagttgctgctcgaaaggcgttccagagctggcgagatgtccccggtgaggagcgtgccaagttgctggacaacttcatcaatctggtgtccaagaacctcgacacggtggctgccatcgaggctctcgattccggcaagcctcttcagctcaatgctcggggtgacatcgccggcggcctggccgtctacaagtactacgcagggtgggcggacaaggtgtttggtaagaccattgtcaacaccaccaagaagctggcgtacactcttcacgagccccatggagtgtgtggtcagatcattccctggaactatccgtttctgatggccgcgtggaagattgcgcctgcaattgcggctggcaacgtggtggtgatgaagctcgcggaaaacacccctctgtcgatgctgtatctgtgcaatctgttcaaggaggccgggttccctcccggagtgatcaacatcttcactggccacggcgccaaggctggctcgcgactggctgagcacccggatgtcgacaagattgccttcaccggctccaccgccaccggccgaatcatcatgaagctggccgctaccaacctcaaggccatcactctggaatgtggaggcaagtcgcccatgattgttctgggagatgccgatctcgaccaggccaccaaatgggcccatgccggtattatgaccaaccagggccagatctgctgcggtgtgtcgcgagtgctggttcacgagtccatctacgaccagtttgtcgacaagtacgtcgaggtggtcaagcagcggtctcgagtcggagacatgttccaggacaagattctccaaggcccccaggtctccaaggtccagcaggagaaggtgcttggctacattgagaagggcaaggaggagggcgccaagctggtctactctggcgctgtggctgccgaggcgctcgaaaagggctactttgtgccccccactgtgtttgctgacgtcagagacgacatggtgatttctcgagaggagattttcggacctgtggttgccatcgccaagttctccgacgtggaagacgccatcaaccgagccaacgactccgagtacggtttggccgcgtccgtctacaccaaggacctgaccgaggcccaccgaatctcccgacggctcgaaagtggccaggtgttcatcaacatggcccatatgggcgactaccgaatgccttttggaggatacaaacagagtggaattggacgagagttgggcgagtatggtctcgatacttatactcagtgcaaggcggtgcatattaacatgggtatgaagttgtag

SEQ ID NO: 7 - DNA, 1569 bp, synthetic construct
  source 1..1569: mol_type = other DNA
atgctccgacgaatcactctcaaccagtttaagggcggcctgcgacggctgtccacccttacccccgtcaagaacgaacctctgaccctgcccaacggcgccaagtacgagcagcccgtcggtctcttcatcaacggcgagttcgtcaagtctcagtccggaaagcgattcgagaccgagaaccccaccaccgagacccccatcatctccgtttacgaggctggtgaggctgatgccaacgcagctgtcgaggctgccaagaatgccttcaagaactggggcttcaagaccgctccttccgagcgaggagtcctgctcaacaagctcgctgatctcattgagcgagatctcgacctcatttctgccattgagaccaccgacaacggtaaggtctttgcccaggcccagggtgacgtcgccctcgtcgtcaaggtgctccgatactacgctggatttgccgacaagattggcggcgacctcgtccagaccaacgacggcttcttcaactacacccgaaaggagcctctcggagtgtgtggccagatcatcccctggaacttccctctgctcatgtgggcctggaagattgcccccgctctgaccactggtaacaccgtggttcttaagaccgccgagtccacccctctgtccgccctgtacgcctgtaagctctcccaggaggctggcttccccaagggtgttctcaacgttgtgtccggttatggccccgttggaggcgttctgtccgcccaccccgacatcaagaagattgctttcaccggctccaccgccactggtaagcaggttgctaagaccgccctgacctccaacctcaagaagaccaccatggagctcggtggtaagtcccccaacattatcttcgacgacgccaacctcgaggacgctctttctgccgccgctctcggtatcttcttcaactccggagaggtctgctgcgccggctctcgactctttgtccaagccggtgtctacgaccaggttgtcgaggccttcaagaagaaggctgagtccgtcaaggtcggtgatcccttcgaccccaactctctccagggtccccagcagaacaagaaccagttcaagaagattctgggatacattgagcagggccagaaggagggcgcccatctcctgtgtggaggatctgcccaggccggtcctaacaagggatacttcatccagcccaccgttttcaccgacgtgaacaacgatatgtccattgtgcgagaggagattttcggccccgtcctgaccatcaccaagttcaacaccgttgacgaagtgattgacatggccaacgactccgagtacggtcttgccgctggtatccacaccactgatatcaacaaggcccactatgttgctgagaacattgcctccggtaccatctgggtcaactgctacaacgccttccacgaggccgttccctttggaggatacaagcagtctggtttcggtaaggagatgggtcgagatggtcttgagaactacctccagaccaaggcagttcgagtcaagcttgatgagcgaaagtgggctgacaagcagtga

SEQ ID NO: 8 - DNA, 1476 bp, synthetic construct
  source 1..1476: mol_type = other DNA
atgcaagttactcttcccgacggaaagtcctacgaccagcccaccaagctcttcatcaacaacgagtgggtcgatggtcacggcggctcaattgagtctgtcaaccccgccaccgagcaggtcatctgctccgttgaggccgctgacgagagtgatgtcgacaaggctgttcaggccgctcgaaactgctacgagaacgtctggcgaaaggtcaccggtgccgagcgagcacagctcatgcgcaagcttgccgaccttgtcgagaagaacaaggacctgctcacctccattgaggctgccgactctggaaagcccaagtacggcaactgtgacggagacgtggacgagctcatctacgtcctgcgatactactccggcctggctgagaaggctggcaatggagtcaccatttctacctccaacgaaaagtttgcctacaccatccacgagccttacggagtctgtggccagatcatcccctggaactaccccattgctatggctgcctggaagctaggtccctgtctcgctgccggtaacgtgctggtcatgaagctttccgaatacacccctctgtccatgctggtcatctgcaacctggtcaaggaggctggtttcccccctggcgtggtgaacgtggttaacggctacggcgccaaggccggcaaccgactggctgagcaccccgacgttgacaagattgccttcaccggttctaccgctaccggtcgatctgtcatgaaggctgctaccggaaacatgaaggccgtgaccatggagcttggaggaaagtctcctctgctcattttcgacgactgcgatctcgccaaggccatcgagtgggcccacattggcatcatgtacaacatgggccaggtgtgttccgccacctctcgaatcctggtgcaagaaggcattgccgacaagttcgtcgagggtttcatcaagcagtgtaatgaggcctccattctgggttgtcctttggaccagaagacctctcacggtcctcaggtcaacaagatccagtatgagaaggtgctcggatacattgagaagggtaaggctgagggagccaagtgcattctgggaggtgaggctgccccccaaaacggcaagggctatttcattaagcccaccgccttcaccaacgtcaacaaggacatgaccatctggaaggaggagattttcggccctgtcgtggtaattgacaccttcaagaccgaggaggaggccattgccaaggccaacgatactccgtacggtctggctgccgctctgtttaccgagaacattcggcgagcccaccgggttgtcaaggagctgcgagctggtcaggtctgggtcaactctgataacgactccgatcctcgagttccctttggtggtgtcaagcagagtggtattggtcgagagcttggtgagtatggtctttctatttacacccaggccaaggccgtccacattaacctggattag

SEQ ID NO: 9 - DNA, 9928 bp, synthetic construct
  source 1..9928: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgcttttcgtagataatggaatacaaatggatatccagagtatacacatggatagtatacactgacacgacaattctgtatctctttatgttaactactgtgaggcgttaaatagagcttgatatataaaatgttacatttcacagtctgaacttttgcagattacctaatttggtaagatattaattatgaactgaaagttgatggcatccctaaatttgatgaaagggcgcgggatccggcccgggaattcgaaggtaccaaggaagcatgcggtacccgaattcctgaggtgtctcacaagtgccgtgcagtcccgcccccacttgcttctctttgtgtgtagtgtacgtacattatcgagaccgttgttcccgcccacctcgatccggtctagactgaggtgtctcacaagtgccgtgcagtcccgcccccacttgcttctctttgtgtgtagtgtacgtacattatcgagaccgttgttcccgcccacctcgatccggggatccctgaggtgtctcacaagtgccgtgcagtcccgcccccacttgcttctctttgtgtgtagtgtacgtacattatcgagaccgttgttcccgcccacctcgatccgggtcgacctgaggtgtctcacaagtgccgtgcagtcccgcccccacttgcttctctttgtgtgtagtgtacgtacattatcgagaccgttgttcccgcccacctcgatccgggagctcctgaggtgtctcacaagtgccgtgcagtcccgcccccacttgcttctctttgtgtgtagtgtacgtacattatcgagaccgttgttcccgcccacctcgatccggtctagactgaggtgtctcacaagtgccgtgcagtcccgcccccacttgcttctctttgtgtgtagtgtacgtacattatcgagaccgttgttcccgcccacctcgatccggggatccctgaggtgtctcacaagtgccgtgcagtcccgcccccacttgcttctctttgtgtgtagtgtacgtacattatcgagaccgttgttcccgcccacctcgatccgggtcgacctgaggtgtctcacaagtgccgtgcagtcccgcccccacttgcttctctttgtgtgtagtgtacgtacattatcgagaccgttgttcccgcccacctcgatccgggcatgcggtacccgaattcctgaggtgtctcacaagtgccgtgcagtcccgcccccacttgcttctctttgtgtgtagtgtacgtacattatcgagaccgttgttcccgcccacctcgatccggtctagactgaggtgtctcacaagtgccgtgcagtcccgcccccacttgcttctctttgtgtgtagtgtacgtacattatcgagaccgttgttcccgcccacctcgatccggggatccctgaggtgtctcacaagtgccgtgcagtcccgcccccacttgcttctctttgtgtgtagtgtacgtacattatcgagaccgttgttcccgcccacctcgatccgggtcgacctgaggtgtctcacaagtgccgtgcagtcccgcccccacttgcttctctttgtgtgtagtgtacgtacattatcgagaccgttgttcccgcccacctcgatccgggagctcctgaggtgtctcacaagtgccgtgcagtcccgcccccacttgcttctctttgtgtgtagtgtacgtacattatcgagaccgttgttcccgcccacctcgatccggtctagactgaggtgtctcacaagtgccgtgcagtcccgcccccacttgcttctctttgtgtgtagtgtacgtacattatcgagaccgttgttcccgcccacctcgatccggggatccctgaggtgtctcacaagtgccgtgcagtcccgcccccacttgcttctctttgtgtgtagtgtacgtacattatcgagaccgttgttcccgcccacctcgatccgggtcgacctgaggtgtctcacaagtgccgtgcagtcccgcccccacttgcttctctttgtgtgtagtgtacgtacattatcgagaccgttgttcccgcccacctcgatccgggcatgcctgcagaagcttagagaccgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaaggcgcgccatgtcagagttcgattgggagtcaattttgccggcaacaccactaggtgagatcgagaaggatattcaaaccctacgacagggcttcaggtccggaaagacgctggatttgaacttcaggcttgaccagattcgtaagcttttctatgctctctatgataatgtcgatgcgatcaaagaagcaattcataaggatctcggacgtccggtcttcgagactgaactttgcgagatctcctttcagtggggtgaattcaataatgtcgtttctaacttgaagaaatgggcagctgatgagacggtgaagggaaccaccattcaatacactctcacccggccaaagattagaaagcgtccacttggtaccgtccttatcatatctccttggaactacccatttgttctgaccatctctcccctgcttgctgctctagcggcaggaaatacggtggccctaaagttctccgaaatgtgcccacatacatcgcttttgctgggaaagttgtgcacagaggcacttgataaagaaattttcaaggcatttcagggaggcgttccggtagtgtcggagattctcaagtacaagttcgacaaaatcatgtacactggaaatcatcgagttggcaagatcatcttggacgcagctaacaaatacctcacccccgttattttggagcttggaggcaaatcaccagtcttcgtgactaagaattgccaaaacgtatctcttgctgccaagcgtgctctgtggggtaaactggtcaacgctggacaaacatgcgttgcccccgattacatcatcgtcgagcctgaggtcgaacaggagtttatcaaagcttgccagtactgggttgagaagttctaccgaggtggagttgactctgatcataaggacttcactcatattgcaacacctggacattggagacgattgacatccatgcttgcccagacagagggaaatatcatcacaggcggaaattcggacgagaaatcacggtttcttgctcccacagttgttgcgaaagttcctgatggtgattctttgatgaatgatgagatctttggccctatcctgcccatcctgacagccagatccgttgacgaaggtattcgctatgttcatgagaatcacgacactcccctggccatgtatgtctttactgataatgcatcagaaggagagtatatccaatctcaaatcaactcaggtggcctgatattcaatgatagtcttgttcacgttggctgcgtgcaggcgccttttggtggtgtcggccaatccggctatgggtcttatcacggcgaagattccttcttggctttttcacacaggcagactttcatgaagcagccccatttcatcgaacgaccaatggcgatcagatatgccccctacactagtcgaaaacaaaaggctgtccagggtagtctagctgctccatcttttcctcgaacaggaaaggttgaccgctccctgttggagcggatatttggtaagctatggttctgggtgatcgttttagggctaggagcagccagtttgaagtcaggaattttcttatgattaattaaggcacgtgcctaaaaaggatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcggctaacttgtttaaacaactgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccagatcctctagagtcgaagcggccgctatgtctgataaaaggatgtaacataggcaagctgctcgtgagtgttgagtacgaaccttagatccaaatcacccgcacccacggatatacttgcttgaatatacagtagtatgcggccgcttcgacaccatatcatataaaactaacaatgcattgcttattacgaagactacccgttgctatctccacaccgttatctccacggtccaaaggctgctcaatgtgctgcatacgtaacgtggggtgcaaccttgagcacatagtacttttccgaaaaccggcgataattaagtgtgcactccaacttttcacactgagcgtaaaatgtggagaagaaatcggcactaaaaagtcaggtagactggaaaatgcgccatgaaatgaatatctcttgctacagtaatgcccagcatcgaggggtattgtgtcaccaacactatagtggcagctgaagcgctcgtgattgtagtatgagtctttattggtgatgggaagagttcactcaatattctcgttactgccaaaacaccacggtaatcggccagacaccatggatgtagatcaccaagcctgtgaatgttattcgagctaaaatgcacatggttggtgaaaggagtagttgctgtcgaattccgtcgtcgcctgagtcatcatttatttaccagttggccacaaacccttgacgatctcgtatgtcccctccgacatactcccggccggctggggtacgttcgatagcgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgtttgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttcctgaggttgagcgaattgcccgaatggccgccttcctggcccttcagcacaacccccctcttcccgtgtggtctcttgacaaggccaacgtgctggcctcctctcgactttggcgaaagactgtcactcgagtcctcaaggacgaattcccccagctcgagctcaaccaccagctgatcgactcggccgccatgatcctcatcaagcagccctccaagatgaatggtatcatcatcaccaccaacatgtttggcgatatcatctccgacgaggcctccgtcatccccggttctctgggtctgctgccctccgcctctctggcttctctgcccgacaccaacgaggcgttcggtctgtacgagccctgtcacggatctgcccccgatctcggcaagcagaaggtcaaccccattgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagacttgttgccaacaaggtcaaggagctgctcaagaaggagtaagtcgtttctacgacgcattgatggaaggagcaaactgacgcgcctgcgggttggtctaccggcagggtccgctagtgtataagactctataaaaagggccctgccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaagctggcgactgcacccaacgagtgtggtggtagcttgttactgtatattcggtaagatatattttgtggggttttagtggtgtttggtaggttagtgcttggtatatgagttgtaggcatgacaatttggaaaggggtggactttgggaatattgtgggatttcaataccttagtttgtacagggtaattgttacaaatgatacaaagaactgtatttcttttcatttgttttaattggttgtatatcaagtccgttagacgagctcagtgccatggcttttggcactgtatttcatttttagaggtacactacattcagtgaggtatggtaaggttgagggcataatgaaggcaccttgtactgacagtcacagacctctcaccgagaattttatgagatatactcgggttcattttaggctccgattcgattcaaattattactgtcgaaatcggttgagcatccgttgatttccgaacagatctggccctttcgtc

SEQ ID NO: 10 - DNA, 8469 bp, synthetic construct
  source 1..8469: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgcttttcgtagataatggaatacaaatggatatccagagtatacacatggatagtatacactgacacgacaattctgtatctctttatgttaactactgtgaggcgttaaatagagcttgatatataaaatgttacatttcacagtctgaacttttgcagattacctaatttggtaagatattaattatgaactgaaagttgatggcatccctaaatttgatgaaagggcgcgggatccggcccgggaattcgaaggtaccaaggaagcatgcggtacccgaattcctgaggtgtctcacaagtgccgtgcagtcccgcccccacttgcttctctttgtgtgtagtgtacgtacattatcgagaccgttgttcccgcccacctcgatccggtctagactgaggtgtctcacaagtgccgtgcagtcccgcccccacttgcttctctttgtgtgtagtgtacgtacattatcgagaccgttgttcccgcccacctcgatccggggatccctgaggtgtctcacaagtgccgtgcagtcccgcccccacttgcttctctttgtgtgtagtgtacgtacattatcgagaccgttgttcccgcccacctcgatccgggtcgacctgaggtgtctcacaagtgccgtgcagtcccgcccccacttgcttctctttgtgtgtagtgtacgtacattatcgagaccgttgttcccgcccacctcgatccgggagctcctgaggtgtctcacaagtgccgtgcagtcccgcccccacttgcttctctttgtgtgtagtgtacgtacattatcgagaccgttgttcccgcccacctcgatccggtctagactgaggtgtctcacaagtgccgtgcagtcccgcccccacttgcttctctttgtgtgtagtgtacgtacattatcgagaccgttgttcccgcccacctcgatccggggatccctgaggtgtctcacaagtgccgtgcagtcccgcccccacttgcttctctttgtgtgtagtgtacgtacattatcgagaccgttgttcccgcccacctcgatccgggtcgacctgaggtgtctcacaagtgccgtgcagtcccgcccccacttgcttctctttgtgtgtagtgtacgtacattatcgagaccgttgttcccgcccacctcgatccgggcatgcggtacccgaattcctgaggtgtctcacaagtgccgtgcagtcccgcccccacttgcttctctttgtgtgtagtgtacgtacattatcgagaccgttgttcccgcccacctcgatccggtctagactgaggtgtctcacaagtgccgtgcagtcccgcccccacttgcttctctttgtgtgtagtgtacgtacattatcgagaccgttgttcccgcccacctcgatccggggatccctgaggtgtctcacaagtgccgtgcagtcccgcccccacttgcttctctttgtgtgtagtgtacgtacattatcgagaccgttgttcccgcccacctcgatccgggtcgacctgaggtgtctcacaagtgccgtgcagtcccgcccccacttgcttctctttgtgtgtagtgtacgtacattatcgagaccgttgttcccgcccacctcgatccgggcatgcctgcagaagcttagagaccgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaaggcgcgccatgactatcgactcacaatactacaagtcgcgagacaaaaacgacacggcacccaaaatcgcgggaatccgatatgccccgctatcgacaccattactcaaccgatgtgagaccttctctctggtctggcacattttcagcattcccactttcctcacaattttcatgctatgctgcgcaattccactgctctggccatttgtgattgcgtatgtagtgtacgctgttaaagacgactccccgtccaacggaggagtggtcaagcgatactcgcctatttcaagaaacttcttcatctggaagctctttggccgctacttccccataactctgcacaagacggtggatctggagcccacgcacacatactaccctctggacgtccaggagtatcacctgattgctgagagatactggccgcagaacaagtacctccgagcaatcatctccaccatcgagtactttctgcccgccttcatgaaacggtctctttctatcaacgagcaggagcagcctgccgagcgagatcctctcctgtctcccgtttctcccagctctccgggttctcaacctgacaagtggattaaccacgacagcagatatagccgtggagaatcatctggctccaacggccacgcctcgggctccgaacttaacggcaacggcaacaatggcaccactaaccgacgacctttgtcgtccgcctctgctggctccactgcatctgattccacgcttcttaacgggtccctcaactcctacgccaaccagatcattggcgaaaacgacccacagctgtcgcccacaaaactcaagcccactggcagaaaatacatcttcggctaccacccccacggcattatcggcatgggagcctttggtggaattgccaccgagggagctggatggtccaagctctttccgggcatccctgtttctcttatgactctcaccaacaacttccgagtgcctctctacagagagtacctcatgagtctgggagtcgcttctgtctccaagaagtcctgcaaggccctcctcaagcgaaaccagtctatctgcattgtcgttggtggagcacaggaaagtcttctggccagacccggtgtcatggacctggtgctactcaagcgaaagggttttgttcgacttggtatggaggtcggaaatgtcgcccttgttcccatcatggcctttggtgagaacgacctctatgaccaggttagcaacgacaagtcgtccaagctgtaccgattccagcagtttgtcaagaacttccttggattcacccttcctttgatgcatgcccgaggcgtcttcaactacgatgtcggtcttgtcccctacaggcgacccgtcaacattgtggttggttcccccattgacttgccttatctcccacaccccaccgacgaagaagtgtccgaataccacgaccgatacatcgccgagctgcagcgaatctacaacgagcacaaggatgaatatttcatcgattggaccgaggagggcaaaggagccccagagttccgaatgattgagtaagttaattaaggcacgtgcctaaaaaggatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcggctaacttgtttaaacaactgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccagatcctctagagtcgaagcggccgctatgtctgataaaaggatgtaacataggcaagctgctcgtgagtgttgagtacgaaccttagatccaaatcacccgcacccacggatatacttgcttgaatatacagtagtatgcggccgcttcgacaccatatcatataaaactaacaatgcatgtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtcatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacaccaaaaccatgatcaaaccacccatggacttcctggaggcagaagaacttgttatggaaaagctcaagagagagaagccaagatactatcaagacatgtgtcgcaacttcaaggaggaccaagctctgtacaccgagaaacaggcctttgtcgacagatctggccctttcgtc

SEQ ID NO: 11 - DNA, 8362 bp, synthetic construct
  source 1..8362: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgcttttcgtagataatggaatacaaatggatatccagagtatacacatggatagtatacactgacacgacaattctgtatctctttatgttaactactgtgaggcgttaaatagagcttgatatataaaatgttacatttcacagtctgaacttttgcagattacctaatttggtaagatattaattatgaactgaaagttgatggcatccctaaatttgatgaaagggcgcgggatccggcccgggaattcgaaggtaccaaggaagcatgcggtacccgaattcctgaggtgtctcacaagtgccgtgcagtcccgcccccacttgcttctctttgtgtgtagtgtacgtacattatcgagaccgttgttcccgcccacctcgatccggtctagactgaggtgtctcacaagtgccgtgcagtcccgcccccacttgcttctctttgtgtgtagtgtacgtacattatcgagaccgttgttcccgcccacctcgatccggggatccctgaggtgtctcacaagtgccgtgcagtcccgcccccacttgcttctctttgtgtgtagtgtacgtacattatcgagaccgttgttcccgcccacctcgatccgggtcgacctgaggtgtctcacaagtgccgtgcagtcccgcccccacttgcttctctttgtgtgtagtgtacgtacattatcgagaccgttgttcccgcccacctcgatccgggagctcctgaggtgtctcacaagtgccgtgcagtcccgcccccacttgcttctctttgtgtgtagtgtacgtacattatcgagaccgttgttcccgcccacctcgatccggtctagactgaggtgtctcacaagtgccgtgcagtcccgcccccacttgcttctctttgtgtgtagtgtacgtacattatcgagaccgttgttcccgcccacctcgatccggggatccctgaggtgtctcacaagtgccgtgcagtcccgcccccacttgcttctctttgtgtgtagtgtacgtacattatcgagaccgttgttcccgcccacctcgatccgggtcgacctgaggtgtctcacaagtgccgtgcagtcccgcccccacttgcttctctttgtgtgtagtgtacgtacattatcgagaccgttgttcccgcccacctcgatccgggcatgcggtacccgaattcctgaggtgtctcacaagtgccgtgcagtcccgcccccacttgcttctctttgtgtgtagtgtacgtacattatcgagaccgttgttcccgcccacctcgatccggtctagactgaggtgtctcacaagtgccgtgcagtcccgcccccacttgcttctctttgtgtgtagtgtacgtacattatcgagaccgttgttcccgcccacctcgatccggggatccctgaggtgtctcacaagtgccgtgcagtcccgcccccacttgcttctctttgtgtgtagtgtacgtacattatcgagaccgttgttcccgcccacctcgatccgggtcgacctgaggtgtctcacaagtgccgtgcagtcccgcccccacttgcttctctttgtgtgtagtgtacgtacattatcgagaccgttgttcccgcccacctcgatccgggagctcctgaggtgtctcacaagtgccgtgcagtcccgcccccacttgcttctctttgtgtgtagtgtacgtacattatcgagaccgttgttcccgcccacctcgatccggtctagactgaggtgtctcacaagtgccgtgcagtcccgcccccacttgcttctctttgtgtgtagtgtacgtacattatcgagaccgttgttcccgcccacctcgatccggggatccctgaggtgtctcacaagtgccgtgcagtcccgcccccacttgcttctctttgtgtgtagtgtacgtacattatcgagaccgttgttcccgcccacctcgatccgggtcgacctgaggtgtctcacaagtgccgtgcagtcccgcccccacttgcttctctttgtgtgtagtgtacgtacattatcgagaccgttgttcccgcccacctcgatccgggcatgcctgcagaagcttagagaccgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaaggcgcgccttaattaaggcacgtgcctaaaaaggatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcggctaacttgtttaaacaactgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccagatcctctagagtcgaagcggccgctatgtctgataaaaggatgtaacataggcaagctgctcgtgagtgttgagtacgaaccttagatccaaatcacccgcacccacggatatacttgcttgaatatacagtagtatgcggccgcttcgacaccatatcatataaaactaacaatgcattgcttattacgaagactacccgttgctatctccacaccgttatctccacggtccaaaggctgctcaatgtgctgcatacgtaacgtggggtgcaaccttgagcacatagtacttttccgaaaaccggcgataattaagtgtgcactccaacttttcacactgagcgtaaaatgtggagaagaaatcggcactaaaaagtcaggtagactggaaaatgcgccatgaaatgaatatctcttgctacagtaatgcccagcatcgaggggtattgtgtcaccaacactatagtggcagctgaagcgctcgtgattgtagtatgagtctttattggtgatgggaagagttcactcaatattctcgttactgccaaaacaccacggtaatcggccagacaccatggatgtagatcaccaagcctgtgaatgttattcgagctaaaatgcacatggttggtgaaaggagtagttgctgtcgaattccgtcgtcgcctgagtcatcatttatttaccagttggccacaaacccttgacgatctcgtatgtcccctccgacatactcccggccggctggggtacgttcgatagcgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgtttgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttcctgaggttgagcgaattgcccgaatggccgccttcctggcccttcagcacaacccccctcttcccgtgtggtctcttgacaaggccaacgtgctggcctcctctcgactttggcgaaagactgtcactcgagtcctcaaggacgaattcccccagctcgagctcaaccaccagctgatcgactcggccgccatgatcctcatcaagcagccctccaagatgaatggtatcatcatcaccaccaacatgtttggcgatatcatctccgacgaggcctccgtcatccccggttctctgggtctgctgccctccgcctctctggcttctctgcccgacaccaacgaggcgttcggtctgtacgagccctgtcacggatctgcccccgatctcggcaagcagaaggtcaaccccattgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagacttgttgccaacaaggtcaaggagctgctcaagaaggagtaagtcgtttctacgacgcattgatggaaggagcaaactgacgcgcctgcgggttggtctaccggcagggtccgctagtgtataagactctataaaaagggccctgccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaagctggcgactgcacccaacgagtgtggtggtagcttgttactgtatattcggtaagatatattttgtggggttttagtggtgtttggtaggttagtgcttggtatatgagttgtaggcatgacaatttggaaaggggtggactttgggaatattgtgggatttcaataccttagtttgtacagggtaattgttacaaatgatacaaagaactgtatttcttttcatttgttttaattggttgtatatcaagtccgttagacgagctcagtgccatggcttttggcactgtatttcatttttagaggtacactacattcagtgaggtatggtaaggttgagggcataatgaaggcaccttgtactgacagtcacagacctctcaccgagaattttatgagatatactcgggttcattttaggctccgattcgattcaaattattactgtcgaaatcggttgagcatccgttgatttccgaacagatctggccctttcgtc